(19) United States
(12) Patent Application Publication
(10) Pub. No.: US 2024/0420817 A1
(43) Pub. Date: Dec. 19, 2024

(54) METHOD AND SYSTEM FOR RECOMMENDING ALTERNATIVES TO BIOLOGICS

(71) Applicant: Tata Consultancy Services Limited, Mumbai (IN)

(72) Inventors: Avishek HALDER, Kolkata (IN); Ninad Yeshwant RAJADHYAKSHA, Mumbai (IN); Smita Elayath VASUDEVAN, Kochi (IN); Amit SAXENA, Gurgaon (IN); Sarang Subhash PURANIK, Bangalore (IN)

(73) Assignee: Tata Consultancy Services Limited, Mumbai (IN)

(21) Appl. No.: 18/741,937

(22) Filed: Jun. 13, 2024

(30) Foreign Application Priority Data
Jun. 16, 2023 (IN) 202321041074

(51) Int. Cl. G16H 20/10; G16H 10/60; G16H 50/70; G16H 70/40

(52) U.S. Cl. CPC G16H 20/10; G16H 10/60; G16H 50/70; G16H 70/40

(57) ABSTRACT

High cost of biotherapy drug makes it unaffordable for patients to seek treatments. Further, access to information related to new low-cost alternatives like Biosimilars and Interchangeable may not be available with the physician at the time of consultation. Most of the conventional approaches aims to select an alternative biosimilar for a reference drug without considering patient's information. The present disclosure recommends a list of low-cost alternatives to high-cost reference drugs thereby enabling the physician to get timely and updated information on development of Biosimilars. The solution leverages Natural Language Processing (NLP) technology to extract known adverse events for a reference drug and a relative scoring based technique to identify and optimum alternative to prescribed biologics. The capability of the solution is further extended to identify secondary adverse events due to multiple drugs, thereby providing a clinical decision support system to help physicians take an informed decision.

### PRIORITY CLAIMS

This application claims priority under 35 U.S.C. § 119 to: India application No. 202321041074, filed on Jun. 16, 2023. The entire contents of the aforementioned application are incorporated herein by reference.

### TECHNICAL FIELD

The disclosure herein generally relates to the field of healthcare and, more particularly, to a method and system for recommending alternatives to biologics.

### BACKGROUND

Biotherapies are a crucial therapeutic option for many patients/subjects with debilitating inflammatory diseases, autoimmune or chronic conditions. With increasing Food and Drug Association (FDA) approved biologics, more Adverse Drug Reactions (ADR) are likely to occur along with higher cost. Further, potency, specificity and effectiveness of biotherapy are constantly evolving with continuous research and development of biological agents. Adverse reaction to biotherapy is a pressing challenge across all stakeholders in the healthcare industry. Furthermore, biotherapies entail incremental costs that have a considerable financial impact on healthcare industry stakeholders like patients, members, payors, providers, and pharmaceutical companies.

Further, high cost of biotherapy/reference drug makes it unaffordable for patients to seek treatments influencing the medical adherence of such patients. Access to information related to new low-cost alternatives like biosimilars and interchangeable drugs may not be available with the physician at the time of consultation, and this is an opportunity for saving costs. Also, access to industrial and academic research related to probable ADRs is not available with the physician at the time of consultation. In certain circumstances, treatment decisions may lead to rehospitalization. Especially for aged or young population who when subjected to polypharmacy, there are very limited options to identify secondary adverse reactions due to the multiple drugs coming into the picture. It is a reactive approach in the industry.

Most of the conventional approaches aims to select an alternative biosimilar for a reference drug. Here, the patient's information is not considered while selecting an alternative biosimilar drug. Further, in most of the conventional approaches, drug reactions are identified based on the interaction between the drug and the protein. Hence there is a need for an approach to identify alternatives to biologics by considering patient's information and to identify ADRs, thereby recommending optimum alternatives for prescribed biologics.

### SUMMARY

Embodiments of the present disclosure present technological improvements as solutions to one or more of the above-mentioned technical problems recognized by the inventors in conventional systems. For example, in one embodiment, a method for recommending alternatives to biologics is provided. The method includes receiving, by one or more hardware processors, at least one prescribed biologics associated with a subject, wherein the at least one prescribed biologics is extracted from an e-Prescription associated with the subject. Further, the method includes extracting by the one or more hardware processors, a plurality of metadata pertaining to the subject from a repository comprising medical details of subjects under medication, wherein the plurality of metadata comprises genetic information, previous diseases and disorders, and a plurality of drugs currently used by the subject and a price associated with the prescribed biologics. Furthermore, the method includes identifying by the one or more hardware processors, a plurality of primary adverse drug reactions (ADRs) and a plurality of diseases associated with the at least one prescribed biologics based on the plurality of metadata pertaining to the subject and the at least one prescribed biologics using a text mining technique, wherein the text mining is performed on an associated database comprising a plurality of biomedical and life sciences research literature. Furthermore, the method includes extracting by the one or more hardware processors, a plurality of reference biologic drugs functionally similar to the at least one prescribed biologics from a Food and Drug Association (FDA) approved biologics list. Furthermore, the method includes creating by the one or more hardware processors, a reference drug database associated with the at least one prescribed biologics based on a plurality of attributes, wherein the plurality of attributes comprises the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs. Furthermore, the method includes identifying by the one or more hardware processors, at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user. Furthermore, the method includes selecting by the one or more hardware processors, a plurality of primary genes responsible for the efficacy of the identified optimum drug from a pharmacogenomic database.

Furthermore, the method includes computing by the one or more hardware processors, a gene-gene connectivity score corresponding to each of the plurality of primary genes using a gene-gene connectivity score computation tool. Furthermore, the method includes identifying by the one or more hardware processors, a plurality of secondary genes interacting with each of the plurality of primary genes using the gene-gene connectivity score computation tool. Furthermore, the method includes identifying by the one or more hardware processors, a plurality of secondary ADRs due to interaction among the plurality of primary genes and the plurality of secondary genes with plurality of drugs currently used by the subject using a gene database. Furthermore, the method includes creating by the one or more hardware processors, a structural database based on the gene-gene connectivity scores associated with the interactions among the plurality of primary genes and the plurality of secondary genes, the interactions among the plurality of drugs currently used by the subject with the plurality of primary genes and the plurality of secondary genes, the plurality of secondary ADRs. Finally, the method includes generating by the one or more hardware processors, a recommendation comprising the identified optimum drug, genes responsible for its efficacy, secondary ADRs occurring due to reaction between the plurality of drugs currently used by the subject and, an associated primary and secondary gene product using the structural database.

In another aspect, a system for recommending alternatives to biologics is provided. The system includes at least one memory storing programmed instructions, one or more Input/Output (I/O) interfaces, and one or more hardware processors operatively coupled to the at least one memory, wherein the one or more hardware processors are configured by the programmed instructions to receive at least one prescribed biologics associated with a subject, wherein the at least one prescribed biologics is extracted from an e-Prescription associated with the subject. Further, the one or more hardware processors are configured by the programmed instructions to extract a plurality of metadata pertaining to the subject from a repository comprising medical details of subjects under medication, wherein the plurality of metadata comprises genetic information, previous diseases and disorders, and a plurality of drugs currently used by the subject and a price associated with the prescribed biologics. Furthermore, the one or more hardware processors are configured by the programmed instructions to identify a plurality of primary adverse drug reactions (ADRs) and a plurality of diseases associated with the at least one prescribed biologics based on the plurality of metadata pertaining to the subject and the at least one prescribed biologics using a text mining technique, wherein the text mining is performed on an associated database comprising a plurality of biomedical and life sciences research literature. Furthermore, the one or more hardware processors are configured by the programmed instructions to extract a plurality of reference biologic drugs functionally similar to the at least one prescribed biologics from a Food and Drug Association (FDA) approved biologics list. Furthermore, the one or more hardware processors are configured by the programmed instructions to create a reference drug database associated with the at least one prescribed biologics based on a plurality of attributes, wherein the plurality of attributes comprises the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs. Furthermore, the one or more hardware processors are configured by the programmed instructions to identify at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user. Furthermore, the one or more hardware processors are configured by the programmed instructions to select a plurality of primary genes responsible for the efficacy of the identified optimum drug from a pharmacogenomic database. Furthermore, the one or more hardware processors are configured by the programmed instructions to compute a gene-gene connectivity score corresponding to each of the plurality of primary genes using a gene-gene connectivity score computation tool. Furthermore, the one or more hardware processors are configured by the programmed instructions to identify a plurality of secondary genes interacting with each of the plurality of primary genes using the gene-gene connectivity score computation tool. Furthermore, the one or more hardware processors are configured by the programmed instructions to identify a plurality of secondary ADRs due to interaction among the plurality of primary genes and the plurality of secondary genes with plurality of drugs currently used by the subject using a gene database. Furthermore, the one or more hardware processors are configured by the programmed instructions to create a structural database based on the gene-gene connectivity scores associated with the interactions among the plurality of primary genes and the plurality of secondary genes, the interactions among the plurality of drugs currently used by the subject with the plurality of primary genes and the plurality of secondary genes, the plurality of secondary ADRs. Finally, one or more hardware processors are configured by the programmed instructions to generate a recommendation comprising the identified optimum drug, genes responsible for its efficacy, secondary ADRs occurring due to reaction between the plurality of drugs currently used by the subject and, an associated primary and secondary gene product using the structural database.

In yet another aspect, a computer program product including a non-transitory computer-readable medium having embodied therein a computer program for recommending alternatives to biologics is provided. The computer readable program, when executed on a computing device, causes the computing device to receive at least one prescribed biologics associated with a subject, wherein the at least one prescribed biologics is extracted from an e-Prescription associated with the subject. Further, the computer readable program, when executed on a computing device, causes the computing device to extract a plurality of metadata pertaining to the subject from a repository comprising medical details of subjects under medication, wherein the plurality of metadata comprises genetic information, previous diseases and disorders, and a plurality of drugs currently used by the subject and a price associated with the prescribed biologics. Furthermore, the computer readable program, when executed on a computing device, causes the computing device to identify a plurality of primary adverse drug reactions (ADRs) and a plurality of diseases associated with the at least one prescribed biologics based on the plurality of metadata pertaining to the subject and the at least one prescribed biologics using a text mining technique, wherein the text mining is performed on an associated database comprising a plurality of biomedical and life sciences research literature. Furthermore, the computer readable program, when executed on a computing device, causes the computing device to extract a plurality of reference biologic drugs functionally similar to the at least one prescribed biologics from a Food and Drug Association (FDA) approved biologics list. Furthermore, the computer readable program, when executed on a computing device, causes the computing device to create a reference drug database associated with the at least one prescribed biologics based on a plurality of attributes, wherein the plurality of attributes comprises the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs. Furthermore, the computer readable program, when executed on a computing device, causes the computing device to identify at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user. Furthermore, computer readable program, when executed on a computing device, causes the computing device to select a plurality of primary genes responsible for the efficacy of the identified optimum drug from a pharmacogenomic database. Furthermore, computer readable program, when executed on a computing device, causes the computing device to compute a gene-gene connectivity score corresponding to each of the plurality of primary genes using a gene-gene connectivity score computation tool. Furthermore, computer readable program, when executed on a computing device, causes the computing device to identify a plurality of secondary genes interacting with each of the plurality of primary genes using the gene-gene connectivity score computation tool. Furthermore, computer readable program, when executed on a computing device, causes the computing device to identify a plurality of secondary ADRs due to interaction among the plurality of primary genes and the plurality of secondary genes with plurality of drugs currently used by the subject using a gene database. Furthermore, computer readable program, when executed on a computing device, causes the computing device to create a structural database based on the gene-gene connectivity scores associated with the interactions among the plurality of primary genes and the plurality of secondary genes, the interactions among the plurality of drugs currently used by the subject with the plurality of primary genes and the plurality of secondary genes, the plurality of secondary ADRs. Finally, computer readable program, when executed on a computing device, causes the computing device to generate a recommendation comprising the identified optimum drug, genes responsible for its efficacy, secondary ADRs occurring due to reaction between the plurality of drugs currently used by the subject and, an associated primary and secondary gene product using the structural database.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 is a functional block diagram of a system for recommending alternatives to biologics, in accordance with some embodiments of the present disclosure.

FIG. 2 illustrates a functional architecture of the system of FIG. 1, for recommending alternatives to biologics, in accordance with some embodiments of the present disclosure.

FIG. 3A and FIG. 3B (collectively called as FIG. 3) is an exemplary flow diagram illustrating a processor implemented method for recommending alternatives to biologics implemented by the system of FIG. 1 according to some embodiments of the present disclosure.

FIG. 4 is an exemplary flow diagram identifying at least one optimum drug for the processor implemented method for recommending alternatives to biologics implemented by the system of FIG. 1 according to some embodiments of the present disclosure.

### DETAILED DESCRIPTION

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments.

High cost of biotherapy/reference drug makes it unaffordable for patients to seek treatments influencing the medical adherence of such patients. Access to information related to new low-cost alternatives like biosimilars and interchangeable drugs may not be available with the physician at the time of consultation. Also, access to industrial and academic research related to probable ADRs is not available to the physician at the time of consultation. Treatment decisions may lead to rehospitalization. Especially, for aged or young population who when subjected to polypharmacy, there are very limited options to identify secondary adverse reactions due to the multiple drugs coming into the picture. It is a reactive approach in the industry.

Most of the conventional approaches aims to select an alternative biosimilar for a reference drug. Here, the patient's information is not considered while selecting an alternative biosimilar drug. Further, most of the conventional approaches drug reactions are identified based on the interaction between the drug and the protein. Hence there is a need for an approach to identify alternatives to biologics by considering patient's information and to identify ADRs, thereby recommending optimum alternatives for prescribed biologics.

To overcome the challenges of the conventional approaches, embodiments herein provide a relative score based method and system for recommending alternatives to biologics. The present disclosure enables the physician to get timely and updated information on the development of Biosimilars or Interchangeable. The present disclosure leverages Natural Language Processing (NLP) technology to extract known adverse events for a reference drug from publicly available validated unstructured research database. Further, the present disclosure implements a relative scoring technique for recommending alternative reference biologic drug to a prescribed biologic drug. The capability of the solution is further extended to identify secondary adverse events/reactions due to multiple drugs, thereby providing a clinical decision support system to help physicians take an informed decision. The present disclosure can also recommend a list of low-cost alternatives (biosimilars and interchangeable drugs) to high-cost Reference drugs

### (Biologics).

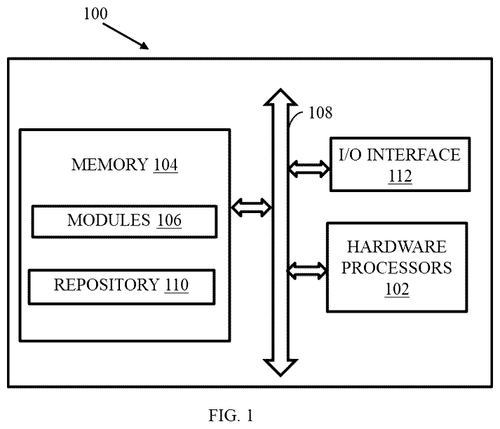

Referring now to the drawings, and more particularly to FIGS. 1 through 4, where similar reference characters denote corresponding features consistently throughout the figures, there are shown preferred embodiments and these embodiments are described in the context of the following exemplary system and/or method.

FIG. 1 is a functional block diagram of a system 100 for recommending alternatives to biologics, in accordance with some embodiments of the present disclosure. The system 100 includes or is otherwise in communication with hardware processors 102, at least one memory such as a memory 104, an I/O interface 112. The hardware processors 102, memory 104, and the Input/Output (I/O) interface 112 may be coupled by a system bus such as a system bus 108 or a similar mechanism. In an embodiment, the hardware processors 102 can be one or more hardware processors.

The I/O interface 112 may include a variety of software and hardware interfaces, for example, a web interface, a graphical user interface, and the like. The I/O interface 112 may include a variety of software and hardware interfaces, for example, interfaces for peripheral device(s), such as a keyboard, a mouse, an external memory, a printer and the like. Further, the I/O interface 112 may enable the system 100 to communicate with other devices, such as web servers, and external databases.

The I/O interface 112 can facilitate multiple communications within a wide variety of networks and protocol types, including wired networks, for example, local area network (LAN), cable, etc., and wireless networks, such as Wireless LAN (WLAN), cellular, or satellite. For the purpose, the I/O interface 112 may include one or more ports for connecting several computing systems with one another or to another server computer. The I/O interface 112 may include one or more ports for connecting several devices to one another or to another server.

The one or more hardware processors 102 may be implemented as one or more microprocessors, microcomputers, microcontrollers, digital signal processors, central processing units, node machines, logic circuitries, and/or any devices that manipulate signals based on operational instructions. Among other capabilities, the one or more hardware processors 102 is configured to fetch and execute computer-readable instructions stored in the memory 104.

The memory 104 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. In an embodiment, the memory 104 includes a plurality of modules 106. The memory 104 also includes a data repository (or repository) 110 for storing data processed, received, and generated by the plurality of modules 106.

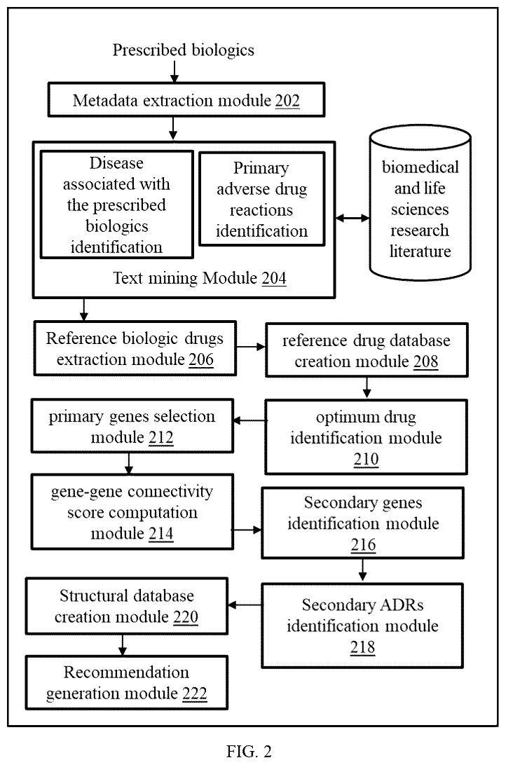

The plurality of modules 106 include programs or coded instructions that supplement applications or functions performed by the system 100 for recommending alternatives to biologics. The plurality of modules 106, amongst other things, can include routines, programs, objects, components, and data structures, which performs particular tasks or implement particular abstract data types. The plurality of modules 106 may also be used as, signal processor(s), node machine(s), logic circuitries, and/or any other device or component that manipulates signals based on operational instructions. Further, the plurality of modules 106 can be used by hardware, by computer-readable instructions executed by the one or more hardware processors 102, or by a combination thereof. The plurality of modules 106 can include various sub-modules (not shown). The plurality of modules 106 may include computer-readable instructions that supplement applications or functions performed by the system 100 for recommending alternatives to biologics. In an embodiment, the modules 106 include a metadata extraction module (shown in FIG. 2), a text mining module (shown in FIG. 2), a reference biologic drugs extraction module (shown in FIG. 2), a reference drug database creation module (shown in FIG. 2), an optimum drug identification module (shown in FIG. 2), a primary genes selection module (shown in FIG. 2), a gene-gene connectivity score computation module (shown in FIG. 2), a secondary genes identification module (shown in FIG. 2), a secondary ADRs identification module (shown in FIG. 2), a structural database creation module (shown in FIG. 2) and a recommendation generation module (shown in FIG. 2). In an embodiment, FIG. 2 illustrates a functional architecture of the system of FIG. 1, for recommending alternatives to biologics, in accordance with some embodiments of the present disclosure.

The data repository (or repository) 110 may include a plurality of abstracted piece of code for refinement and data that is processed, received, or generated as a result of the execution of the plurality of modules in the module(s) 106.

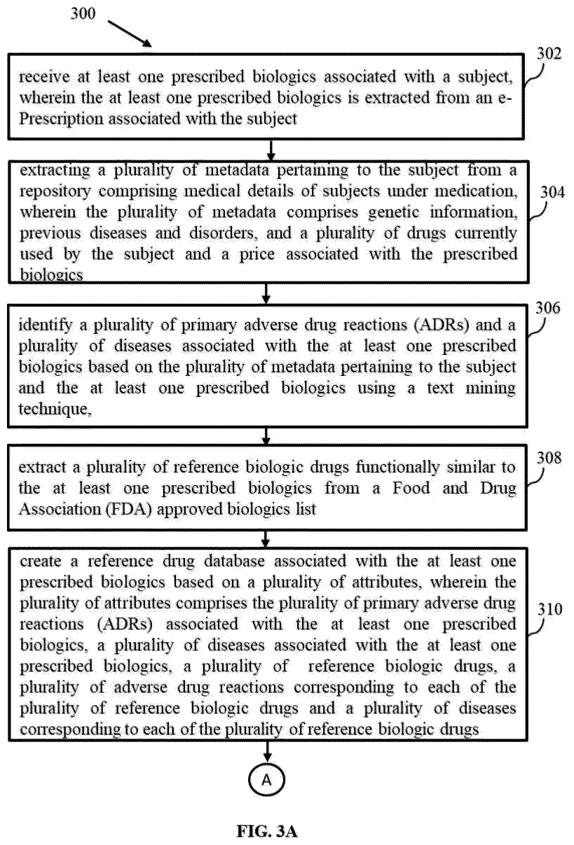

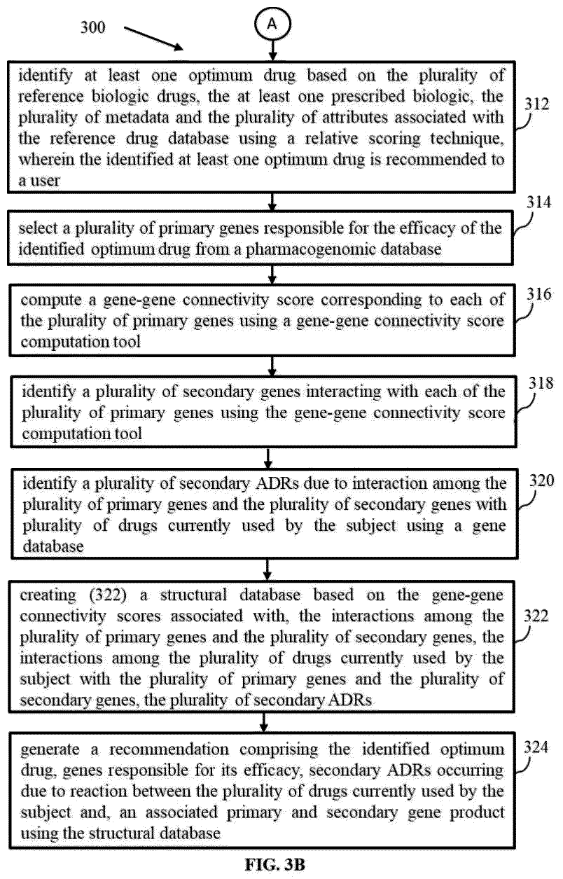

Although the data repository 110 is shown internal to the system 100, it will be noted that, in alternate embodiments, the data repository 110 can also be implemented external to the system 100, where the data repository 110 may be stored within a database (repository 110) communicatively coupled to the system 100. The data contained within such an external database may be periodically updated. For example, new data may be added into the database (not shown in FIG. 1) and/or existing data may be modified and/or non-useful data may be deleted from the database. In one example, the data may be stored in an external system, such as a Lightweight Directory Access Protocol (LDAP) directory and a Relational Database Management System (RDBMS). The working of the components of the system 100 are explained with reference to the method steps depicted in FIG. 3, FIG. 6A and FIG. 6B.

FIG. 3A and FIG. 3B (collectively called as FIG. 3) is an exemplary flow diagram illustrating a method 300 for recommending alternatives to biologics implemented by the system of FIG. 1 according to some embodiments of the present disclosure. In an embodiment, the system 100 includes one or more data storage devices or the memory 104 operatively coupled to the one or more hardware processor(s) 102 and is configured to store instructions for execution of steps of the method 300 by the one or more hardware processors 102. The steps of the method 300 of the present disclosure will now be explained with reference to the components or blocks of the system 100 as depicted in FIG. 1 and the steps of flow diagram as depicted in FIG. 3. The method 300 may be described in the general context of computer executable instructions. Generally, computer executable instructions can include routines, programs, objects, components, data structures, procedures, modules, functions, etc., that perform particular functions or implement particular abstract data types. The method 300 may also be practiced in a distributed computing environment where functions are performed by remote processing devices that are linked through a communication network. The order in which the method 300 is described is not intended to be construed as a limitation, and any number of the described method blocks can be combined in any order to implement the method 300, or an alternative method. Furthermore, the method 300 can be implemented in any suitable hardware, software, firmware, or combination thereof.

At step 302 of the method 300, the one or more hardware processors 102 are configured by the programmed instructions to receive at least one prescribed biologics associated with a subject, wherein the at least one prescribed biologics is extracted from an e-Prescription associated with the subject. For example, the subject is a patient undergoing some treatment. Biologics are drugs prepared from living organisms or including components of living organisms.

At step 304 of the method 300, the metadata extraction module 202 executed by one or more hardware processors 102 is configured by the programmed instructions to extract a plurality of metadata pertaining to the subject from a repository comprising medical details of subjects under medication. The plurality of metadata includes genetic information, previous diseases and disorders, and a plurality of drugs currently used by the subject and a price associated with the prescribed biologics.

At step 306 of the method 300, the text mining module 204 executed by the one or more hardware processors 102 is configured by the programmed instructions to identify a plurality of primary adverse drug reactions (ADRs) and a plurality of diseases associated with the at least one prescribed biologics based on the plurality of metadata pertaining to the subject and the at least one prescribed biologics using a text mining technique. The text mining is performed in an associated database including a plurality of biomedical and life sciences research literature. In an embodiment, the text mining is performed using PubMed® database.

For example, the literature documents related to Humira drug (adalimumab) are taken. Further, text like “Adverse reactions”, “Side-Effects”, “Cytokine overstimulation”, “Allergic reactions” are used to search the ADRs of the drug. In an embodiment, spaCy library from python is used for this approach. spaCy library is a python-based NLP framework. Using spaCy the text in the literature is converted into containers like Doc, Span and token. Further, a custom pipeline is created to define custom entities, add Entity ruler and Regex patterns to match the above strings like “Adverse reactions”. The spaCy also provides a vector similar strings of these string like “Adverse reactions” which are also included in the regex pattern. Based on the matches received from the regex pattern matching the ADR can be detected.

### Example 1

Using Spacy library-first apply a pattern for the Keywords like Pattern

=[{“LOWER”:”adverse”},{“IS_PUNCT”:True,’OP’:’?’},{“LOWER”:”drug”,’OP’
:’?’},
{“IS_PUNCT”:True,’OP’:’?’},{“LOWER”:”reaction”,’OP’:’?’}]

Applying this pattern would give around 40 sentences across all the pages in the Pdf.
Further for each matched sentence—
   For each token in the sentence-retrieve the token text, token Dependency and Token parts of speech (POS).
   Add a logic to check where the token dependency is Subject and token text is “Adverse drug reactions” and the sentence has POS like AUX like are, ADJ like Such or ADP like as—then filter that sentence.
   On applying this logic, we get the result as 1 like below

“The most common adverse drug reactions (ADR) associated with ETN use are infections and injection site reactions.”

### Example 2—Eliminating the False Positives

Apply pattern for keyword like “Hpersensitive reaction”, “Adverse Effects” “Adalimumab”, “Anti-TNF”

```
Pattern=[{“LOWER”:”adverse”},{“IS_PUNCT”:True,’OP’:’?’},{“LOWER”:”ef
  fects”,’OP’:’?’},
  {“IS_PUNCT”:True,’OP’:’?’},{“LOWER”:”Hypersensitive”,’OP’:’?’},
  {“IS_PUNCT”:True,’OP’:’?’},{“LOWER”:”reaction”,’OP’:’?’},{“LOW
  ER”:” Adalimumab”,’OP’:’?’},{“LOWER”:” Anti-TNF”,’OP’:’?’}]
```

Got a result of around 12 sentences.
These sentences are further tokenized to analyze the token dependency and Token parts of speech.
A logic is added to compare the Dependency to be any of the pattern elements along with the drug names to be amod(Modifier) or nObj.
This logic filters down to the following 2 statements.

“An increases frequency of severe infections as well as reactivation of tuberculosis and HBV hepatitis are well known adverse effects of anti-TNF agents and have been discussed in reviews”.

At step 308 of the method 300, the reference biologic drugs extraction module 206 executed by the one or more hardware processors 102 is configured by the programmed instructions to extract a plurality of reference biologic drugs functionally similar to the at least one prescribed biologics from a Food and Drug Association (FDA) approved biologics list. For example, considering the drug “Humira (adalimumab)”, the plurality of reference drugs is “Orencia (abatacept)” and “Actemra (tocilizumab)”.

At step 310 of the method 300, the reference drug database creation module 208 executed by the one or more hardware processors 102 is configured by the programmed instructions to create a reference drug database associated with the at least one prescribed biologics based on a plurality of attributes. The plurality of attributes includes the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs. An example reference drug database is shown in Table 1.

TABLE 1

| | | | | | Diseases |
|---|---|---|---|---|---|
| | | | | | associated |
| | | | | ADRs of | with the |
| | | | Reference | reference | reference |
| Prescribed | Primary | Associated | biologic | biologic | biologic |
| biologic | ADRs | diseases | drugs | drugs | drugs |
| Humira | adalimumab | Rheumatoid | Orencia | Bacterial | Rheumatoid |
| (adalimumab) | optic | arthritis | (abatacept) | infection, | arthritis, |
| | neuropathy, | | | | Spondylarthritis |
| | bacterial | | Actemra | Infection | Rheumatoid |
| | and | | (tocilizumab) | related | arthritis, |
| | mycobacterial | | | headache, | giant cell |
| | infection, | | | hypertension, | arthritis |
| | etc. | | | increased | |
| | | | | liver | |
| | | | | enzymes, | |
| | | | | injection | |
| | | | | site | |
| | | | | reactions | |

At step 312 of the method 300, the optimum drug identification module_210 executed by the one or more hardware processors 102 is configured by the programmed instructions to identify at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user. For example, the user can be a subject undergoing medication, a member, a payer and a provider.

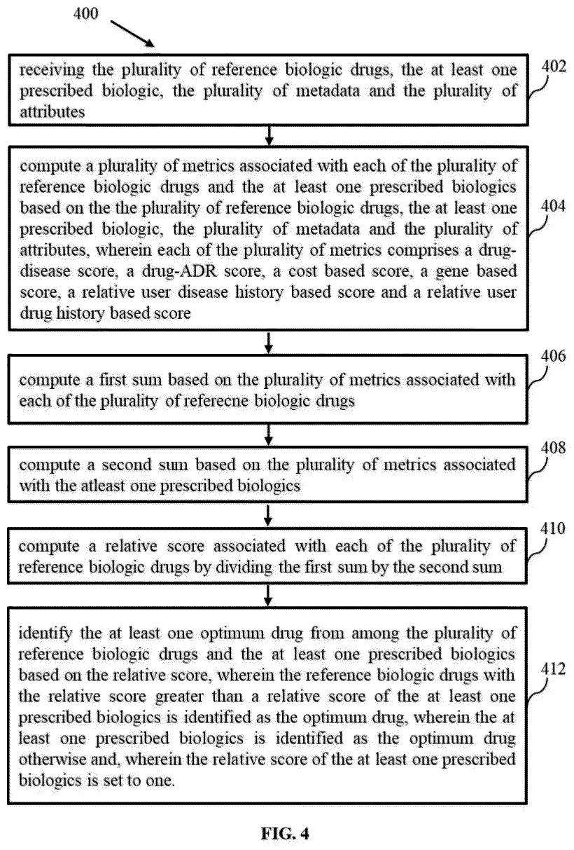

In an embodiment, the steps involved in identifying the at least one optimum drug is explained in reference to FIG. 4. Now referring to FIG. 4, the plurality of reference biologic drugs, the at least one prescribed biologic, the plurality of metadata and the plurality of attributes are received initially at step 402. Further, at step 404, a plurality of metrics associated with each of the plurality of reference biologic drugs and the at least one prescribed biologics are computed based on the plurality of reference biologic drugs, the at least one prescribed biologic, the plurality of metadata and the plurality of attributes. In an embodiment, the plurality of metrics includes a drug-disease score, a drug-ADR score, a cost based score, a gene based score, a relative user disease history based score and a relative user drug history based score.

In an embodiment, the drug-disease score corresponding to each of the plurality of reference biologic drugs is generated based on a capability to address a number of diseases. For example, a reference biologic drug addressing one disease is assigned a score of one, wherein a reference biologic drug addressing two diseases is assigned a score of two and the like.

In an embodiment, the drug-ADR score corresponding to each of the plurality of reference biologic drugs is generated based on number of ADRs associated with each of the plurality of reference biologic drugs. For example, a reference drug with no ADR is assigned a score of zero, wherein a reference drug with one ADR is assigned a score of negative one, wherein a reference drug with two ADRs is assigned a score of negative two and the like.

In an embodiment, the cost based score corresponding to each of the plurality of reference biologic drugs is generated based on a plurality of price ranges. For example, the most appropriate costs of approved Biologics are available with Payers Industry. The price of the Prescribed drug or the reference drug is obtained and a score of positive one is assigned if the price of the reference biologic is less than the prescribed biologic, wherein a score of zero will be assigned to a reference biologic if it's cost is equal to the cost of the prescribed drug, wherein a score of negative one will be assigned to the reference biologic if it's cost is higher than the cost of the prescribed biologic.

In an embodiment, the gene based score corresponding to each of the plurality of reference biologic drugs is proportional to a range of genetic mutation associated with each of the plurality of reference biologic drugs. A subject undergoing medication having higher genetic mutation will correspond to lower score. For example, there are one hundred and eleven genes responsible for the efficacy of the biologic drug called Humira. The top five genes are TNF (Tumor Necrosis Factor), TNFRSF1A ((Tumor Necrosis Factor Receptor Super Family member 1A), FCGR2A (Fibrosis Cystic Gamma Receptor 2A), FCGR3A (Fibrosis Cystic Gamma Receptor 3A), KLRC1 (Killer Cell Lectin Like Receptor C1). In this situation, for a subject who has no mutation will be assigned a score of zero, for a subject with mutation on one gene will be assigned a score of negative one, for a subject with mutations on two genes will be assigned a score of negative two, and the like.

In an embodiment, the relative user disease history based score is proportional to a number of previous diseases of the subject. For example, a diabetic person under medication for breast cancer (Her2 positive case), the person will be taking insulin (Hormone) and trastuzumab (monoclonal antibody against Her2). They are totally different classes of biologics with different mode of actions. Therefore, there would be a disease history for cancer and a drug history for cancer. However, cancer is not related to diabetes so the score for disease will be zero. However, for a subject with diabetes and diabetic related rheumatic arthritis the patient will be using insulin and Humira, respectively. These diseases are related, and one will induce influence on the other, So, the score will be negative one, and on the like.

In an embodiment, the relative user drug history based score is proportional to a number of other drugs consumed by a patient currently. For example, a diabetic person under medication for breast cancer (Her2 positive case), the person will be taking biologicals like insulin (Hormone) and trastuzumab (monoclonal antibody against Her2) for diabetes and cancer, respectively. They are totally different classes of biologics with different mode of actions. Therefore, there would be a disease history for cancer and a drug history for cancer but trastuzumab is not related to insulin so the score for drug will be zero. However, for a subject with diabetes and diabetic related rheumatic arthritis the patient will be using insulin and Humira, respectively. These drugs will induce influence on the other, So, the score will be negative one, and the like.

Now referring back to FIG. 4, at step 406, a first sum is computed based on the plurality of metrics associated with each of the plurality of reference biologic drugs. At step 408, a second sum is computed based on the plurality of metrics associated with the at least one prescribed biologics. At step 410, a relative score associated with each of the plurality of reference biologic drugs is computed by dividing the first sum by the second sum. Finally, at step 412, the at least one optimum drug is identified from among the plurality of reference biologic drugs and the at least one prescribed biologics based on the relative score. The reference biologic drugs with the relative score greater than a relative score of the at least one prescribed biologics is identified as the optimum drug and the at least one prescribed biologics is identified as the optimum drug otherwise. In an embodiment, the relative score of the at least one prescribed biologics is set to one.

Now referring back to FIG. 3, at step 314 of the method 300, the primary genes selection module 212 executed by the one or more hardware processors 102 is configured by the programmed instructions to selecting a plurality of primary genes responsible for the efficacy of the identified optimum drug from a pharmacogenomic database. In an embodiment, PharmGKB is used for selecting primary genes. For example, there are one hundred and eleven genes responsible for the efficacy of the biologic drug called Humira. The top five genes are TNF, TNFRSF1A, FCGR2A, FCGR3A, KLRC1 and there will be rest also available for consideration, using such pharmacogenomic tools.

At step 316 of the method 300, the gene-gene connectivity score computation module 214 executed by the one or more hardware processors 102 is configured by the programmed instructions to compute a gene-gene connectivity score corresponding to each of the plurality of primary genes using a gene-gene connectivity score computation tool. In an embodiment, GUILDify tool had been developed to prioritize gene-disease relationships and identify disease modules. GUILDiFy tool can be used for creation of interactome of protein-protein or gene-gene connectivity score computation. For example, when the names of the primary genes are provided as inputs in the GuildiFy V2.0 the tool will provide names and UniProt IDs of multiple genes associated with a score known as GUILD (Gene Underlying Inheritance Linked Disorders) score. A GUILD score is assigned to each protein in the interactome based on the genes/protein used as the seed. The higher the score, the more likely that an association exists between the genes/protein and the set of seeds (Primary genes) used to expand.

At step 318 of the method 300, the secondary genes identification module_216 executed by the one or more hardware processors 102 is configured by the programmed instructions to identify a plurality of secondary genes interacting with each of the plurality of primary genes using the gene-gene connectivity score computation tool. In an embodiment, only those genes with GUILD score of greater than zero were considered as secondary genes. For example, the secondary genes responsible for the efficacy of the biologic Humira are CD274 (GUILDiFy score 0.3583), ADAMTS1 (GUILDiFy score 0.3264, etc.

At step 320 of the method 300, the secondary ADRs identification module_218 executed by the one or more hardware processors 102 is configured by the programmed instructions to identify a plurality of secondary ADRs due to interaction among the plurality of primary genes and the plurality of secondary genes with plurality of drugs currently used by the subject using a gene database. For example, one of the genes responsible for efficacy of Humira is IL2 but for a patient who is prescribed with Humira and Ciprofloxacin can show adverse reactions like increased Lacrimation. These kinds of adverse events are referred to as secondary adverse events.

At step 322 of the method 300, the structural database creation module 220 executed by the one or more hardware processors 102 is configured by the programmed instructions to create a structural database based on the gene-gene connectivity scores associated with, the interactions among the plurality of primary genes and the plurality of secondary genes, the interactions among the plurality of drugs currently used by the subject with the plurality of primary genes and the plurality of secondary genes, the plurality of secondary ADRs. An example structural database is shown in Table 2.

TABLE 2

| | | | Other drug | | |
|---|---|---|---|---|---|
| | | | consumed | Gene- | |
| | Primary | Secondary | by the | drug | Secondary |
| Biologic | Genes | genes | Subject | interaction | ADR |
| Humira | KLRC1 | CD274 | Ciprofloxacin | — | — |
| | | (GS = | | | |
| | | 0.3583) | | | |
| | TNF | ADAMTS1 | — | — | — |
| | | A (GS = | | | |
| | | 0.3265) | | | |
| | IL2 | PGF (GS = | | Positive | Increased |
| | | 0.3265) | | | Lacrimation |
| | TNFRSF1 | | — | — | — |
| | A | | | | |

At step 324 of the method 300, the recommendation generation module_222 executed by the one or more hardware processors 102 is configured by the programmed instructions to generate a recommendation comprising the identified optimum drug, genes responsible for its efficacy, secondary ADRs occurring due to reaction between the plurality of drugs currently used by the subject and, an associated primary and secondary gene product using the structural database. For example, the recommendation is shown in Table 3. The interpretation of Table 3 is “the analysis shows potential adverse events like increased lacrimation for the subject against Ciprofloxacin, Cyclosporin and the likes, when combined with Humira”.

TABLE 3

| | | | Other | | |
|---|---|---|---|---|---|
| | | | drugs | | |
| | | | consumed | Gene- | |
| Optimal | Primary | Secondary | by the | drug | Secondary |
| drug | Genes | genes | Subject | interaction | ADR |
| Humira | KLRC1 | CD274 | Ciprofloxacin | | |
| (For | | (GS = | | | |
| exam- | | 0.3583) | | | |
| ple) | TNF | ADAMTS1 | | | |
| | | A (GS = | | | |
| | | 0.3265) | | | |
| | IL2 | PGF (GS = | | Positive | Increased |
| | | 0.3265) | | | Lacrimation |
| | TNFRSF1 | | | | |
| | A | | | | |

In an embodiment, the present disclosure generates a plurality of low cost biotherapy based on the at least one prescribed drug from an associated database. For example, United States of America's Food and Drug Administration (FDA) maintains Purple Book database which includes information on availability of low-cost biosimilars or Interchangeable for any biologic. For a reference product like Humira there are seven biosimilars like Abrilada, Amjevita, Hadlima, Hulio, Hyrimoz, Idacio, Yusimry and one interchangeable like Cyltezo.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments of present disclosure herein address the unresolved problem of recommending alternatives to biologics. The present disclosure provides a relative scoring based approach to identify alternatives for prescribed biologics considering treatment-effectiveness in tandem with affordability and accessibility of therapy. In addition, the present disclosure enables the process of detection of possible adverse effects of either any Biotherapy or due to polypharmacy. This supports the physicians or the payers to make informed decision on the medication.

It is to be understood that the scope of the protection is extended to such a program and in addition to a computer-readable means having a message therein such computer-readable storage means contain program-code means for implementation of one or more steps of the method when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software modules located therein. Thus, the means can include both hardware means and software means. The method embodiments described herein could be implemented in hardware and software. The device may also include software means. Alternatively, the embodiments may be implemented on different hardware devices, e.g. using a plurality of CPUs, GPUs and edge computing devices.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device. The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e. non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor implemented method comprising:
receiving, by one or more hardware processors, at least one prescribed biologics associated with a subject, wherein the at least one prescribed biologics is extracted from an e-Prescription associated with the subject;
extracting, by the one or more hardware processors, a plurality of metadata pertaining to the subject from a repository comprising medical details of subjects under medication, wherein the plurality of metadata comprises genetic information, previous diseases and disorders, and a plurality of drugs currently used by the subject and a price associated with the prescribed biologics;
identifying, by the one or more hardware processors, a plurality of primary adverse drug reactions (ADRs) and a plurality of diseases associated with the at least one prescribed biologics based on the plurality of metadata pertaining to the subject and the at least one prescribed biologics using a text mining technique, wherein the text mining is performed on an associated database comprising a plurality of biomedical and life sciences research literature;
extracting, by the one or more hardware processors, a plurality of reference biologic drugs functionally similar to the at least one prescribed biologics from a Food and Drug Association (FDA) approved biologics list;
creating, by the one or more hardware processors, a reference drug database associated with the at least one prescribed biologics based on a plurality of attributes, wherein the plurality of attributes comprises the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs;
identifying, by the one or more hardware processors, at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user;
selecting, by the one or more hardware processors, a plurality of primary genes responsible for the efficacy of the identified optimum drug from a pharmacogenomic database;
computing, by the one or more hardware processors, a gene-gene connectivity score corresponding to each of the plurality of primary genes using a gene-gene connectivity score computation tool;
identifying, by the one or more hardware processors, a plurality of secondary genes interacting with each of the plurality of primary genes using the gene-gene connectivity score computation tool;
identifying, by the one or more hardware processors, a plurality of secondary ADRs due to interaction among the plurality of primary genes and the plurality of secondary genes with plurality of drugs currently used by the subject using a gene database;
creating, by the one or more hardware processors, a structural database based on the gene-gene connectivity scores associated with the interactions among the plurality of primary genes and the plurality of secondary genes, the interactions among the plurality of drugs currently used by the subject with the plurality of primary genes and the plurality of secondary genes, the plurality of secondary ADRs; and
generating, by the one or more hardware processors, a recommendation comprising the identified optimum drug, genes responsible for its efficacy, secondary ADRs occurring due to reaction between the plurality of drugs currently used by the subject and, an associated primary and secondary gene product using the structural database.

2. The processor implemented method of claim 1, wherein a plurality of low cost biotherapy is generated based on the at least one prescribed drug from an associated database.

3. The processor implemented method of claim 1, wherein identifying the at least one optimum drug based on the plurality of reference biologic drugs, the at least one prescribed biologic, the plurality of metadata and the plurality of attributes associated with the reference drug database using the relative scoring technique comprises:
Receiving the plurality of reference biologic drugs, the at least one prescribed biologic, the plurality of metadata and the plurality of attributes;
computing a plurality of metrics associated with each of the plurality of reference biologic drugs and the at least one prescribed biologics based on the plurality of reference biologic drugs, the at least one prescribed biologic, the plurality of metadata and the plurality of attributes, wherein each of the plurality of metrics comprises a drug-disease score, a drug-ADR score, a cost based score, a gene based score, a relative user disease history based score and a relative user drug history based score,
wherein the drug-disease score corresponding to each of the plurality of reference biologic drugs is generated based on a capability to address a number of diseases, wherein a reference biologic drug addressing one diseases is assigned a score of one, wherein a reference biologic drug addressing two diseases is assigned a score of two,
wherein the drug-ADR score corresponding to each of the plurality of reference biologic drugs is generated based on number of ADRs associated with each of the plurality of reference biologic drugs, wherein a reference drug with no ADR is assigned a score of zero, wherein a reference drug with one ADR is assigned a score of negative one and, wherein a reference drug with two ADRs is assigned a score of negative two,
wherein a cost based score corresponding to each of the plurality of reference biologic drugs is generated based on a plurality of price ranges;
wherein the gene based score corresponding to each of the plurality of reference biologic drugs is inversely proportional to a range of genetic mutation associated with each of the plurality of reference biologic drugs, wherein a subject undergoing medication having higher genetic mutation will correspond to lower score,
wherein a relative user disease history based score is proportional to a number of previous disease of the subject, and
wherein the relative user drug history based score is proportional to a number of other drugs consumed by a patient currently;
computing a first sum based on the plurality of metrics associated with each of the plurality of reference biologic drugs;
computing a second sum based on the plurality of metrics associated with the at least one prescribed biologics;
computing a relative score associated with each of the plurality of reference biologic drugs by dividing the first sum by the second sum; and
identifying the at least one optimum drug from among the plurality of reference biologic drugs and the at least one prescribed biologics based on the relative score, wherein the reference biologic drugs with the relative score greater than a relative score of the at least one prescribed biologics is identified as the optimum drug, wherein the at least one prescribed biologics is identified as the optimum drug otherwise, and wherein the relative score of the at least one prescribed biologics is set to one.

4. A system comprising:
at least one memory storing programmed instructions; one or more Input/Output (I/O) interfaces; and one or more hardware processors operatively coupled to the at least one memory, wherein the one or more hardware processors are configured by the programmed instructions to:
receive at least one prescribed biologics associated with a subject, wherein the at least one prescribed biologics is extracted from an e-Prescription associated with the subject;
extract a plurality of metadata pertaining to the subject from a repository comprising medical details of subjects under medication, wherein the plurality of metadata comprises genetic information, previous diseases and disorders, and a plurality of drugs currently used by the subject and a price associated with the prescribed biologics;
identify a plurality of primary adverse drug reactions (ADRs) and a plurality of diseases associated with the at least one prescribed biologics based on the plurality of metadata pertaining to the subject and the at least one prescribed biologics using a text mining technique, wherein the text mining is performed on an associated database comprising a plurality of biomedical and life sciences research literature;
extract a plurality of reference biologic drugs functionally similar to the at least one prescribed biologics from a Food and Drug Association (FDA) approved biologics list;
create a reference drug database associated with the at least one prescribed biologics based on a plurality of attributes, wherein the plurality of attributes comprises the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs;
identify at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user;
select a plurality of primary genes responsible for the efficacy of the identified optimum drug from a pharmacogenomic database;
compute a gene-gene connectivity score corresponding to each of the plurality of primary genes using a gene-gene connectivity score computation tool;
identify a plurality of secondary genes interacting with each of the plurality of primary genes using the gene-gene connectivity score computation tool;
identify a plurality of secondary ADRs due to interaction among the plurality of primary genes and the plurality of secondary genes with plurality of drugs currently used by the subject using a gene database;
create a structural database based on the gene-gene connectivity scores associated with the interactions among the plurality of primary genes and the plurality of secondary genes, the interactions among the plurality of drugs currently used by the subject with the plurality of primary genes and the plurality of secondary genes, the plurality of secondary ADRs; and
generate a recommendation comprising the identified optimum drug, genes responsible for its efficacy, secondary ADRs occurring due to reaction between the plurality of drugs currently used by the subject and, an associated primary and secondary gene product using the structural database.

5. The system of claim 4, wherein a plurality of low cost biotherapy is generated based on the at least one prescribed drug from an associated database.

6. The system of claim 4, wherein identifying the at least one optimum drug based on the plurality of reference biologic drugs, the at least one prescribed biologic, the plurality of metadata and the plurality of attributes associated with the reference drug database using the relative scoring technique comprises:
receiving the plurality of reference biologic drugs, the at least one prescribed biologic, the plurality of metadata and the plurality of attributes;
computing a plurality of metrics associated with each of the plurality of reference biologic drugs and the at least one prescribed biologics based on the plurality of reference biologic drugs, the at least one prescribed biologic, the plurality of metadata and the plurality of attributes, wherein each of the plurality of metrics comprises a drug-disease score, a drug-ADR score, a cost based score, a gene based score, a relative user disease history based score and a relative user drug history based score,
wherein the drug-disease score corresponding to each of the plurality of reference biologic drugs is generated based on a capability to address a number of diseases, wherein a reference biologic drug addressing one diseases is assigned a score of one, wherein a reference biologic drug addressing two diseases is assigned a score of two,
wherein the drug-ADR score corresponding to each of the plurality of reference biologic drugs is generated based on number of ADRs associated with each of the plurality of reference biologic drugs, wherein a reference drug with no ADR is assigned a score of zero, wherein a reference drug with one ADR is assigned a score of negative one and, wherein a reference drug with two ADRs is assigned a score of negative two,
wherein a cost based score corresponding to each of the plurality of reference biologic drugs is generated based on a plurality of price ranges;
wherein the gene based score corresponding to each of the plurality of reference biologic drugs is inversely proportional to a range of genetic mutation associated with each of the plurality of reference biologic drugs, wherein a subject undergoing medication having higher genetic mutation will correspond to lower score,
wherein a relative user disease history based score is proportional to a number of previous disease of the subject, and
wherein the relative user drug history based score is proportional to a number of other drugs consumed by a patient currently;
computing a first sum based on the plurality of metrics associated with each of the plurality of reference biologic drugs;
computing a second sum based on the plurality of metrics associated with the at least one prescribed biologics;
computing a relative score associated with each of the plurality of reference biologic drugs by dividing the first sum by the second sum; and
identifying the at least one optimum drug from among the plurality of reference biologic drugs and the at least one prescribed biologics based on the relative score, wherein the reference biologic drugs with the relative score greater than a relative score of the at least one prescribed biologics is identified as the optimum drug, wherein the at least one prescribed biologics is identified as the optimum drug otherwise, and wherein the relative score of the at least one prescribed biologics is set to one.

7. One or more non-transitory machine-readable information storage mediums comprising one or more instructions which when executed by one or more hardware processors cause:
receiving at least one prescribed biologics associated with a subject, wherein the at least one prescribed biologics is extracted from an e-Prescription associated with the subject;
extracting a plurality of metadata pertaining to the subject from a repository comprising medical details of subjects under medication, wherein the plurality of metadata comprises genetic information, previous diseases and disorders, and a plurality of drugs currently used by the subject and a price associated with the prescribed biologics;
identifying a plurality of primary adverse drug reactions (ADRs) and a plurality of diseases associated with the at least one prescribed biologics based on the plurality of metadata pertaining to the subject and the at least one prescribed biologics using a text mining technique, wherein the text mining is performed on an associated database comprising a plurality of biomedical and life sciences research literature;
extracting a plurality of reference biologic drugs functionally similar to the at least one prescribed biologics from a Food and Drug Association (FDA) approved biologics list;
creating a reference drug database associated with the at least one prescribed biologics based on a plurality of attributes, wherein the plurality of attributes comprises the plurality of primary adverse drug reactions (ADRs) associated with the at least one prescribed biologics, a plurality of diseases associated with the at least one prescribed biologics, a plurality of reference biologic drugs, a plurality of adverse drug reactions corresponding to each of the plurality of reference biologic drugs and a plurality of diseases corresponding to each of the plurality of reference biologic drugs;
identifying at least one optimum drug based on the plurality of reference biologic drugs, the at least one prescribed biologic, the plurality of metadata and the plurality of attributes associated with the reference drug database using a relative scoring technique, wherein the identified at least one optimum drug is recommended to a user;
selecting a plurality of primary genes responsible for the efficacy of the identified optimum drug from a pharmacogenomic database;
computing a gene-gene connectivity score corresponding to each of the plurality of primary genes using a gene-gene connectivity score computation tool;
identifying a plurality of secondary genes interacting with each of the plurality of primary genes using the gene-gene connectivity score computation tool;
identifying a plurality of secondary ADRs due to interaction among the plurality of primary genes and the plurality of secondary genes with plurality of drugs currently used by the subject using a gene database;
creating a structural database based on the gene-gene connectivity scores associated with the interactions among the plurality of primary genes and the plurality of secondary genes, the interactions among the plurality of drugs currently used by the subject with the plurality of primary genes and the plurality of secondary genes, the plurality of secondary ADRs; and
generating a recommendation comprising the identified optimum drug, genes responsible for its efficacy, secondary ADRs occurring due to reaction between the plurality of drugs currently used by the subject and, an associated primary and secondary gene product using the structural database.

8. The one or more non-transitory machine-readable information storage mediums of claim 7, wherein a plurality of low cost biotherapy is generated based on the at least one prescribed drug from an associated database.

9. The one or more non-transitory machine-readable information storage mediums of claim 7, wherein identifying the at least one optimum drug based on the plurality of reference biologic drugs, the at least one prescribed biologic, the plurality of metadata and the plurality of attributes associated with the reference drug database using the relative scoring technique comprises:
receiving the plurality of reference biologic drugs, the at least one prescribed biologic, the plurality of metadata and the plurality of attributes;
computing a plurality of metrics associated with each of the plurality of reference biologic drugs and the at least one prescribed biologics based on the plurality of reference biologic drugs, the at least one prescribed biologic, the plurality of metadata and the plurality of attributes, wherein each of the plurality of metrics comprises a drug-disease score, a drug-ADR score, a cost based score, a gene based score, a relative user disease history based score and a relative user drug history based score,
wherein the drug-disease score corresponding to each of the plurality of reference biologic drugs is generated based on a capability to address a number of diseases, wherein a reference biologic drug addressing one diseases is assigned a score of one, wherein a reference biologic drug addressing two diseases is assigned a score of two,
wherein the drug-ADR score corresponding to each of the plurality of reference biologic drugs is generated based on number of ADRs associated with each of the plurality of reference biologic drugs, wherein a reference drug with no ADR is assigned a score of zero, wherein a reference drug with one ADR is assigned a score of negative one and, wherein a reference drug with two ADRs is assigned a score of negative two,
wherein a cost based score corresponding to each of the plurality of reference biologic drugs is generated based on a plurality of price ranges;
wherein the gene based score corresponding to each of the plurality of reference biologic drugs is inversely proportional to a range of genetic mutation associated with each of the plurality of reference biologic drugs, wherein a subject undergoing medication having higher genetic mutation will correspond to lower score,
wherein a relative user disease history based score is proportional to a number of previous disease of the subject, and
wherein the relative user drug history based score is proportional to a number of other drugs consumed by a patient currently;
computing a first sum based on the plurality of metrics associated with each of the plurality of reference biologic drugs;
computing a second sum based on the plurality of metrics associated with the at least one prescribed biologics;
computing a relative score associated with each of the plurality of reference biologic drugs by dividing the first sum by the second sum; and
identifying the at least one optimum drug from among the plurality of reference biologic drugs and the at least one prescribed biologics based on the relative score, wherein the reference biologic drugs with the relative score greater than a relative score of the at least one prescribed biologics is identified as the optimum drug, wherein the at least one prescribed biologics is identified as the optimum drug otherwise, and wherein the relative score of the at least one prescribed biologics is set to one.
